(19) United States
(12) Patent Application Publication
(10) Pub. No.: US 2017/0312274 A1
(43) Pub. Date: Nov. 2, 2017

(54) METHOD FOR TREATING A BRAIN TUMOUR

(71) Applicant: The Hospital for Sick Children, Toronto (CA)

(72) Inventors: Peter Dirks, Toronto (CA); Sonam Dolma, Toronto (CA)

(21) Appl. No.: 15/526,080

(22) PCT Filed: Nov. 13, 2015
(86) PCT No.: PCT/CA2015/051185

§ 371 Date: May 11, 2017

Related U.S. Application Data
(60) Provisional application No. 62/079,759, filed on Nov. 14, 2014.

(51) Int. Cl. A61K 31/496; A61K 9/08; A61K 9/00; A61K 31/495; A61K 31/454

(52) U.S. Cl. CPC A61K 31/496; A61K 31/495; A61K 31/454; A61K 9/0019; A61K 9/0053; A61K 9/08

(57) ABSTRACT

A method of treating a brain tumour such as glioblastoma in a mammal is provided comprising administering to the mammal a DRD4 antagonist.

### FIELD OF THE INVENTION

The present invention generally relates to the treatment of brain tumours, and more particularly relates to modulation of dopamine receptors to treat brain tumours.

### BACKGROUND OF THE INVENTION

Glioblastoma (GBM) is the most common malignant primary brain tumour in adults and has proven resistant to all therapeutic strategies attempted to date. The median survival time for GBM patients is 15 months even with standard care of treatment including surgery, radiation and chemotherapy. The alkylating agent temozolomide (TMZ) is the only chemotherapeutic of any benefit, and it is effective only transiently in a subset of patients. Long-term treatment with TMZ causes secondary mutations in GBM and increases risks of hematological malignancies. Novel therapeutic approaches based on central nervous system (CNS)-accessible drugs, which might be used in combination with TMZ or other standard treatments, are thus urgently required.

GBM growth is initiated and maintained by small subpopulations of tumourigenic cells termed GBM stem cells, which have a phenotype similar to normal neural stem cells (NSCs). GBM stem cells contribute to tumour progression and resistance to therapy such that long-term disease control is likely to require elimination of this driver population, in addition to the more differentiated tumour bulk. A deeper understanding of the regulatory mechanisms that govern the proliferation and survival of GBM stem cells will be essential to developing rational new therapies.

Neurotransmitters are endogenous chemical messengers that mediate the synaptic function of differentiated neural cells in the mature CNS. Recent studies suggest an important role of neurochemicals, for example gamma-aminobutyric acid (GABA) and glutamate, in regulating NSC fate both in early development and in adult neurogenesis. GABA regulates adult mouse hippocampal NSCs by maintaining their quiescence through the GABA_{A} receptor, yet can also promote embryonic NSC proliferation, suggesting context specific functions. These effects may reflect influences of local or more distant neuronal activity on the NSC niche. Consistent with this idea, dopamine afferents project to neurogenic zones and depletion of dopamine decreases the proliferation of progenitor cells in the adult subventricular zone (SVZ) through D2-like receptors. Dopamine is also present in early neuronal development in the lateral ganglionic eminence (LGE) and modulates LGE progenitor cell proliferation. Serotonin signaling similarly contributes to the SVZ NSC niche.

Neurochemicals and their receptors have also been implicated in the growth and progression of many non-CNS cancers. The mechanisms whereby neurochemicals affect cancer growth are not understood. Thus, it would be desirable to determine if neurochemicals and/or their receptors have an impact on CNS cancers such as brain tumours.

### SUMMARY OF THE INVENTION

It has now been determined that dopamine receptor D4 (DRD4) antagonists exhibit selective growth inhibition of brain tumour stem cells such as glioblastoma stem cells, and thus, are useful to treat brain tumours.

Thus, in one aspect of the invention, a method of treating a brain tumour in a mammal is provided, comprising administering to the mammal a DRD4 antagonist.

In another aspect of the invention, a synergistic composition is provided comprising a DRD4 antagonist in combination with an anti-neoplastic alkylating agent.

These and other aspects of the invention are described herein by reference to the following figures.

### BRIEF DESCRIPTION OF THE FIGURES

FIG. 1. Identification of GNS-selective compounds A. Following a primary screen, compounds showing 20% growth inhibition (5 μM) across the six cell lines screened, secondary screens were done in dose series to determine the fold selectivity (IC₅₀ of BJ/IC₅₀ of any NS or GNS cells with the lowest IC₅₀); B. Percentage of different neurochemical classes enriched in the total hits; C. Percent activity (hits) of each neurochemical class. Number of hits/total number of compounds present in the library of each class; D. The ten NS selective compounds and their IC₅₀ (μM) across different cell lines. Four Non-NS control cell lines in black color, three NS lines in purple color and three GNS lines in pink color. The ten NS selective compounds are grouped under their neurochemical classes;

FIG. 2. DRD4 antagonists selectively inhibit GNS growth and reduce clonogenic cell frequency in primary GBM samples. A. Percent cell viability of 4 Non-NS control cell lines, 3 NS and 6 ONS lines upon treatment with PNU 96415E in dilution series from 0.39 μM-50 μM. Controls n=3 mean±SEM, NS lines n=5-15 mean±SEM, GNS lines n=3-12 mean±SEM; B. Percent cell viability of control BJ fibroblast, 3 NS and 5 GNS lines upon treatment with L-741,742 in dilution series from 0.39 μM-50 μM. BJ n=3 mean±SEM, NS lines n=3-11, mean±SEM, GNS lines n=3-7, mean±SEM; C. Phase contrast image of differential response of GNS (G362) cells and NS (hf5205) cells to L-741,742 (5 μM) treatment after 5 days. Scale bar (100 μm); D. Linear regression plot of in vitro LDA for primary freshly dissociated GBM patient tumour (GBM686) treated with L-741,742 (10 μM), PNU 96415E (25 μM) and DMSO. Frequency of neurosphere forming cells at 95% confidence interval in control DMSO, L-741,742 and PNU 96415E treated cells analyzed using Sigma plot. Representative phase contrast image of neurospheres at day 14 in well seeded with 2000 cells. Scale bar (100 μm);

FIG. 3. DRD4 antagonists inhibit GBM xenograft growth in vivo. A. A schematic of in vivo xenograft experiment in NSG mice. All mice sacrificed at the same end point when tumours reached 17 mm in size in any mouse; B. Growth curve of subcutaneous implanted tumour (G362) over period of time, tumour volume measured from day 12 after implantation when tumours were palpable until the end point when any tumours reached 17 mm in size. Control n=15, mean±SEM, PNU 96415E n=16, mean±SEM, L-741,742 n=16, mean±SEM. **p value <0.005, *p value <0.05 unpaired one-tailed t-test; C. A dot plot showing tumour mass of each tumour from the three treatment groups at end point. Control n=15, mean±SEM, PNU 96415E n=16, mean±SEM, L-741,742 n=16, mean±SEM. Significance analyzed by t-test unpaired one-tailed; D. A representative picture of right and left flank tumour of the same mouse from each group; E. Linear regression plot of in vitro LDA for an in vivo treated tumours. Three tumours from three different mice from each group were dissociated and seeded for in vitro LDA assay. Average of each group was taken for the plot, neurospheres scored for 18 wells (6 wells from each tumour). Frequency of neurosphere forming cells calculated at 95% confidence interval for each group using Sigma plot;

FIG. 4. Primary GBM and GNS cells express functional DRD4 receptor. A. Western blot analysis for anti-DRD4 and anti-β-actin across different NS and GNS lines; B. Western blot analysis for anti-DRD4 and anti-β-actin across different primary GBM patient tumour samples; C. Fold change of cAMP levels in GNS (G362) cells treated with forskolin (30 μM) alone and pretreatment with DRD4 specific agonist A412997 (30 μM) followed by forskolin treatment. N=2, mean±STDEV; D&E. Western blot analysis for anti-DRD4 and anti-β-actin in G362 and G481 cells respectively, transiently transfected with shRNA against DRD4 and eGFP at 72 h post transfection. Band intensity of DRD4 knockdown lanes expressed relative to respective controls; F. Cell viability assay for G362 cells transiently transfected with shRNA-DRD4 and shRNA-eGFP measured over 5 days. N=3, mean±SEM. *p<0.005, **p<0.0005 unpaired one-tailed t-test; G. Cell viability assay for G481 cells transiently transfected with shRNA-DRD4 and shRNA-eGFP measured over 5 days. N=3, mean±SEM.*p<0.005, **p<0.0005 unpaired one-tailed t-test;

FIG. 5. Gene expression profiling revealed accumulation of autophagic vacuoles. A. Gene set enrichment map of pathways containing genes down regulated upon PNU 96415E treatment; B. Gene set enrichment map of pathways containing genes up regulated upon PNU 96415E treatment. Coloured circles (nodes) represent gene-sets (pathways) that were significantly enriched in the comparison treated versus control samples (FDR<=0.002); C. Western blot analysis for anti-LC3B and anti-β actin in GNS cells (G411&G362) treated with PNU 96415E (25 μM) and L-741,742 (10 μM) at indicated time points; D. Immunofluorescence staining for LC3B⁺ punta in GNS cells (G362&G411) treated with PNU 96415E (25 μM) and L-741,742 (10 μM) at 48 h. Scale bar=17 μm. Quantification of LC3B⁺ punta cells in each group (cells counted >200 cells); E. Transmission Electron microscopy images showing large autophagic vacuoles in GNS cells (G362&G411) treated with L-741,742 (10 μM) and PNU 96415E (25 μM) compared to control DMSO at 48 h treatment. Arrows indicate enlarged autophagic vacuoles. Scale bar-100 nm;

FIG. 6. DRD4 antagonism impairs autophagy/lysosomal degradation pathway, causing cell cycle arrest. A. Western blot analysis for anti-LC3B, anti-p62 and anti-β-actin in G411 cells treated with L-741,742 (10 μM) or PNU 96415E (25 μM) in the presence and absence of lysosomal inhibitor chloroquine (30 μM) at 48 h treatment. Western quantification for LC3B-II was done using β-actin as control. N=3, mean±SEM; B. Western blot analysis for corresponding anti-LC3B with anti-p62, anti-LAMP1, anti-mono & poly ubiquitinated protein conjugates (FK2) and anti-β-actin in GNS cells (G411) treated with L-741,742 (10 μM) and PNU 96415E (25 μM) at indicated times points; C. Fluorescence staining of CytoID-Green (autophagosomal marker) and lysoID-Red (lysosomal marker) in live GNS cells (G411&G362) treated with L-741,742 (10 μM) and PNU 96415E (25 μM) at 48 h treatment. White arrows indicate cells stained for autophagic marker CytoID-green and non-colocalization of CytoID and lysoID. Scale bar-15 μm; D&E. Western blot analysis for corresponding anti-DRD4, anti-LC3B, anti-p62, anti-mono and poly ubiquitinated protein conjugate (FK2) and anti-β-actin in transient transfected sh-DRD4 and sh-eGFP G362 and G411 cells post 72 h respectively; F. Cell cycle analysis of GNS cells (G411 and G362) measured by flow cytometry after treatment with L-741,742 (10 μM) and PNU 96415E (25 μM) at 48 h;

FIG. 7. Phospho-kinase array reveals suppression of ERK1/2 pathway upon DRD4 antagonism. A. A dot blot containing 43 phosphoproteins in duplicates after exposure to lysate of G362 cells treated with L-741,742 (10 μM) and PNU 96415E (25 μM) and DMSO for 24 h; B. Signal intensity of each dot spot corresponding to each phosphoprotein (average of two spots for each phosphoprotein) that changed upon treatment with L-741,742 and PNU 96415E compared to DMSO. Signal intensity was quantified using ImageJ; C. Western blot analysis for anti-phospho-ERK1/2 and anti-ERK1/2 in G362 lines treated with L-741,742 (10 μM) at indicated time intervals; D. Western blot analysis for anti-phospho-ERK1/2 and anti-ERK1/2 in G362 cells treated with PNU 96415E (25 μM) and L-741,742 (10 μM) for a longer period of time; E&F. Western blot analysis for anti-phospho-ERK1/2 and anti-ERK½ in G481 and G362 cells transiently transfected with sh-DRD4 and control sh-eGFP post 72 h. (Same protein lysates from FIGS. 4D&E);

FIG. 8. Synergistic effect of DRD4 antagonists with TMZ. A&B. Growth inhibition plot for G362 cells with TMZ in combination with L-741742 or PNU 96415E respectively; C&D. Growth inhibition plot for G481 cells with TMZ in combination with L-741,742 or PNU 96415E respectively; E. Combination index plot for combination of TMZ with L-741742 (L7) or PNU 96415E (PNU) in G362 cells. Combination index (CI) plotted against fractions affected (Fa) analyzed using software COMPUSYN; F. Combination index plot for combination of TMZ with L-741,742 (L7) or PNU 96415E (PNU) in G481 cells; and

FIG. 9 illustrates the nucleic acid-encoding sequence (A) and amino acid sequence (B) of DRD4.

### DETAILED DESCRIPTION OF THE INVENTION

A method of treating a brain tumour in a mammal is provided comprising administering to the mammal a dopamine receptor D₄ antagonist (DRD4).

The term “brain tumour” is used herein to refer to glioblastoma multiforme, also known as grade IV astrocytoma or grade IV glioma; malignant astrocytoma (also called anaplastic astrocytoma, both considered grade III); oligodendroglioma, oligoastrocytoma, mixed glioma and malignant glioma. The brain tumour may be an adult or paediatric form. Brain tumour is also meant to include medulloblastoma.

The term “mammal” is used herein to encompass human and non-human mammals, including domesticated animals such as dogs, cats, horses and the like; and undomesticated animals.

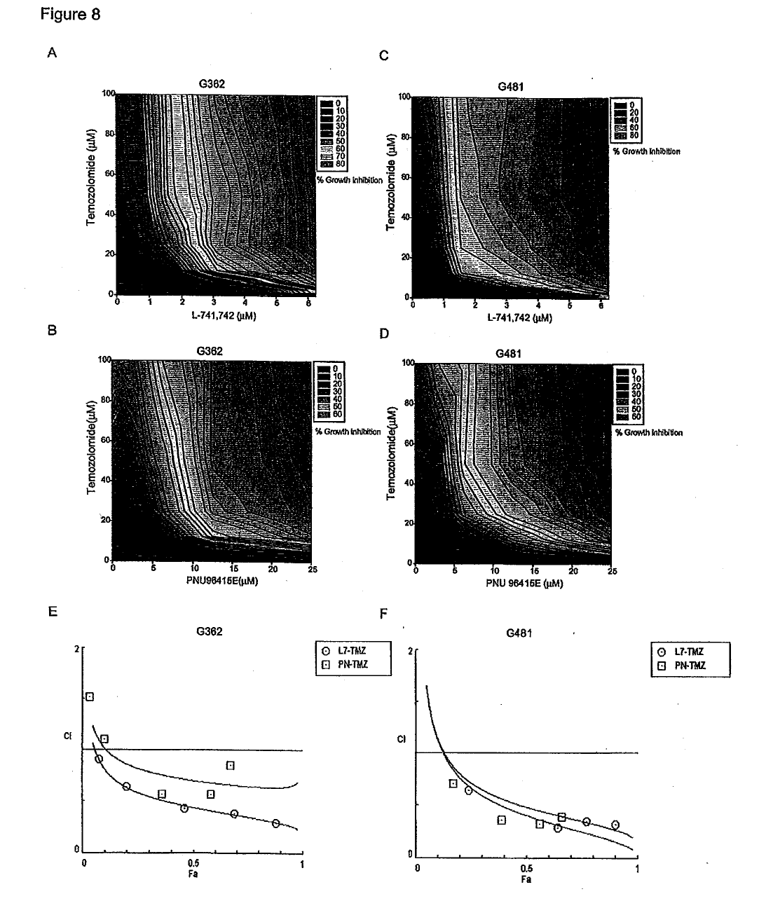

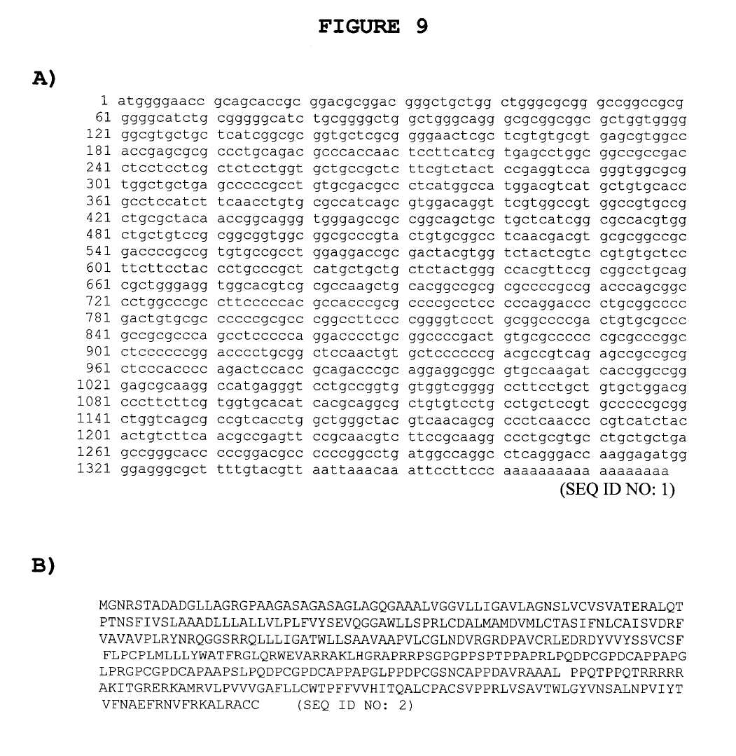

The term “DRD4”, or dopamine receptor D₄, is a G protein-coupled receptor. As with other dopamine receptor subtypes, the D₄ receptor is activated by the neurotransmitter dopamine. The D₄ receptor is D₂-like in that the activated D₄ receptor inhibits the enzyme adenylate cyclase, thereby reducing intracellular cAMP. The D₄ receptor is encoded by the DRD4 gene (e.g. FIG. 9A). As used herein, DRD4 encompasses mammalian DRD4, including the human receptor (FIG. 9B), functionally equivalent variants and isoforms thereof, as well as non-human DRD4, e.g. non-human species such as mouse (FIG. 9C/D), rat, dog, cat, etc. The term “functionally equivalent” refers to variants and isoforms of the DRD4 receptor that essentially retain function as a D4 dopamine receptor, e.g. retain ligand binding activity. The term “functionally equivalent” is used herein to refer to a D₄ receptor protein that exhibits the same or similar function to the native protein (retains at least about 50% of the activity of the human receptor), and includes all isoforms, variants (e.g. Val194Gly), and other naturally-occurring forms. The term “functionally equivalent” also refers to nucleic acid, e.g. mRNA, DNA or cDNA, encoding the D₄ receptor, and is meant to include any nucleic acid sequence that encodes a functional D₄ receptor, including all transcript variants, variants that encode protein isoforms, variants due to degeneracy of the genetic code, and the like. Protein modifications may include, but are not limited to, one or more amino acid substitutions, additions or deletions; modifications to amino acid side chains; and the like. Nucleic acid modifications may include one or more base differences due to degeneracy of the genetic code or sequence differences which encode D₄ variants such as variants incorporating a 48-base pair variable number tandem repeat (VNTR) in exon 3 (e.g. 2-11 repeats), C-521T in the promoter, 13-base pair deletion of bases 235 to 247 in exon 1, 12 base pair repeat in exon I, or a polymorphic tandem duplication of 120 bp.

Antagonists of the dopamine D₄ receptor include compounds that inhibit or prevent the activity of the D₄ receptor, for example, by inhibiting the interaction, such as binding interaction at a binding or active site, of the receptor with its endogenous ligand or substrate. Examples of dopamine D₄ receptor antagonists include, but are not limited to, A-381393, L-745,870, L-750,667, L-741,742, S 18126, fananserin, clozapine, buspirone, FAUC 213, sonepiprazole, PD 168568 dihydrochloride and PNU 96415E. Preferred antagonists include antagonists which are specific for DRD4 such as L-741,742 and PNU 96415E.

As one of skill in the art will appreciate, dopamine D₄ receptor antagonists may be formulated for use to treat a brain tumour in accordance with the present invention. Thus, the selected antagonist may be formulated by combination with a pharmaceutically acceptable carrier. The expression “pharmaceutically acceptable” means acceptable for use in the pharmaceutical and veterinary arts, i.e. not being unacceptably toxic or otherwise unsuitable. As one of skill in the art will appreciate, the selected carrier will vary with the administrable route used. In this regard, the selected antagonist may be administered by any suitable route. In one embodiment, the selected antagonist is formulated for administration by infusion or injection, either subcutaneously or intravenously, and thus, may accordingly be utilized in combination with a medical-grade carrier, such as an aqueous solution in sterile and pyrogen-free form, optionally buffered or made isotonic. Thus, suitable carriers include distilled water or, more desirably, a sterile carbohydrate-containing solution (e.g. sucrose or dextrose) or a sterile saline solution comprising sodium chloride and optionally buffered. Suitable sterile saline solutions may include varying concentrations of sodium chloride, for example, normal saline (0.9%), half-normal saline (0.45%), quarter-normal saline (0.22%), and solutions comprising greater amounts of sodium chloride (e.g. 3%-7%, or greater). Saline solutions may optionally include additional components, e.g. carbohydrates such as dextrose and the like. Examples of saline solutions including additional components, include Ringer's solution, e.g. lactated or acetated Ringer's solution, phosphate buffered saline (PBS), TRIS (hydroxymethyl) aminomethane hydroxymethyl) aminomethane)-buffered saline (TBS), Hank's balanced salt solution (HBSS), Earle's balanced solution (EBSS), standard saline citrate (SSC), HEPES-buffered saline (HBS) and Gey's balanced salt solution (GBSS).

In other embodiments, the selected antagonist may be formulated for administration by routes including, but not limited to, oral, intraperitoneal, intranasal, enteral, topical, sublingual, intramuscular, intra-arterial, intramedullary, intrathecal, inhalation, ocular, transdermal, vaginal or rectal routes, and will be combined with appropriate carriers in each case. For example, compositions for oral administration via tablet, capsule or suspension may be prepared using adjuvants including sugars, such as lactose, glucose and sucrose; starches such as corn starch and potato starch; cellulose and derivatives thereof, including sodium carboxymethylcellulose, ethylcellulose and cellulose acetates; powdered tragancanth; malt; gelatin; talc; stearic acids; magnesium stearate; calcium sulfate; vegetable oils, such as peanut oils, cotton seed oil, sesame oil, olive oil and corn oil; polyols such as propylene glycol, glycerine, sorbital, mannitol and polyethylene glycol; agar; alginic acids; water; isotonic saline and phosphate buffer solutions. Wetting agents, lubricants such as sodium lauryl sulfate, stabilizers, tableting agents, anti-oxidants, preservatives, colouring agents and flavouring agents may also be present. Compositions for topical application may be prepared including appropriate carriers. Creams, lotions and ointments may be prepared for topical application using an appropriate base such as a triglyceride base. Such creams, lotions and ointments may also contain a surface active agent. Aerosol formulations may also be prepared in which suitable propellant adjuvants are used. Other adjuvants may also be added to the composition regardless of how it is to be administered, for example, anti-microbial agents may be added to the composition to prevent microbial growth over prolonged storage periods.

In the present method of treating a brain tumour such as glioblastoma, a dopamine D₄ receptor antagonist is administered to a mammal in need of treatment. The terms “treat”, “treating” or “treatment” are used herein to refer to methods that favorably alter the target pathological condition, i.e. a brain tumour such as a glioblastoma, including those that moderate, reverse, reduce the severity of, or protect against, the progression of glioblastoma. Thus, for use to treat a brain tumour such as glioblastoma, a therapeutically effective amount of a dopamine D₄ receptor antagonist is administered to a mammal in need of treatment. The term “therapeutically effective amount” is an amount of DRD4 antagonist required to treat the tumour, while not exceeding an amount which may cause significant adverse effects. DRD4 antagonist dosages that are therapeutically effective will vary on many factors including the individual being treated and the extent of the disease to be treated. In one embodiment, dosages within the range of about 0.1-100 mg/m² are appropriate for use to treat a brain tumour such as glioblastoma, for example, a dosage in the range of 1-100 mg/m², or 1-50 mg/m².

In an embodiment of the invention, the DRD4 antagonist may be used to treat a tumour such as glioblastoma together with an anti-neoplastic alkylating or alkylating-like agent, i.e. an agent that disrupts DNA (tumour cell DNA), for example by attachment of the agent or an alkyl group from the agent to the DNA, e.g. to the guanine base of DNA at the number 7 nitrogen atom of its purine ring. Examples of such alkylating agents include, but are not limited to, nitrogen mustards such as cyclophosphamide, chlorambucil, uramustine, ifosfamide, melphalan and bendamustinetriazenes; nitrosoureas such as carmustine, lomustine, semustine, ethylnitrosourea (ENU) and streptozocin; alkyl sulfonates such as busulfan; procarbazine, altretamine and triazines such as dacarbazine, mitozolomide and temozolomide; and platinum-based chemotherapeutic agents such as cisplatin, carboplatin, nedaplatin, oxaliplatin, satraplatin and triplatin tetranitrate.

The DRD4 antagonist may be administered in conjunction with an alkylating agent, either together with the alkylating agent or separately, simultaneously or at different times. The use of a DRD4 antagonist with the alkylating agent has been found to have a synergistic effect, i.e. an effect that is greater than the expected additive effect of the DRD4 antagonist and the alkylating agent on a brain tumour such as glioblastoma. The DRD4 antagonist and the alkylating agent may be administered in any suitable administrable form. Preferred routes of administration include orally and by injection. Generally, the dosages of each of the DRD4 antagonist and the alkylating agent will be decreased when used in combination due to the synergy of the combination, in comparison to the dosages of each when used alone to treat a brain tumour. Thus, therapeutically effective dosages of DRD4 antagonist and the alkylating agent for use in a combination treatment include DRD4 antagonist in a dosage range of about 0.1-50 mg/m², for example, 0.5-10 mg/m², such 1-5 mg/m², and the alkylating agent such as temozolomide, in a dosage range of about 1-250 mg/m², for example, 50-150 mg/m², such as 60-80 mg/m², e.g. 75 mg/m², or a dosage of the alkylating agent such as temozolomide which is less than 100 mg/m².

In another aspect of the invention, a synergistic composition is provided comprising a DRD4 antagonist in combination with an antineoplastic alkylating agent such as one of a nitrogen mustard such as cyclophosphamide, chlorambucil, uramustine, ifosfamide, melphalan and bendamustinetriazenes; a nitrosourea such as carmustine, lomustine, semustine, ethylnitrosourea (ENU) and streptozocin; an alkyl sulfonate such as busulfan; procarbazine, altretamine or a triazine such as dacarbazine, mitozolomide and temozolomide; or a platinum-based chemotherapeutic agent such as cisplatin, carboplatin, nedaplatin, oxaliplatin, satraplatin or triplatin tetranitrate. The combination may additionally include pharmaceutically acceptable carriers as described, which are appropriate with respect to the administrable form of the composition. The combination includes suitable dosages of each of the DRD4 antagonist and the alkylating agent also as described.

Embodiments of the present invention are illustrated in the following specific example which is not to be construed as limiting.

### Example 1

### Experimental Procedures

GNS and NS lines were grown as an adherent monolayer culture in serum free medium as described previously (Pollard et al. 2009. Cell stem cell 4, 568-580). Primary tumour cells were freshly dissociated from the patient sample in artificial cerebrospinal fluid followed by treatment with an enzyme cocktail at 37° C. (Singh et al. 2003. Cancer research 63, 5821-5828). BJ fibroblast, Daoy and C8-D1A and U-2 0S (ATCC) were maintained in DMEM with 10% FBS.

### Compound Library

The neurotransmitter library was purchased from BIOMOL international (now integrated into Enzo Life Sciences). The library contains 680 compounds covering thirteen classes of neurochemicals. The compounds were supplied in DMSO at 10 mM concentration in 96-well medium deep plates and stored at −80° C. All compounds for retest were purchased from Tocris Bioscience.

### Chemical Screens

Cells were seeded in laminin-coated 384 well plates at a density of 2000 cells per well. Compounds were added at a concentration of approximately 5 μM and incubated with cells for five days at 37° C. Cell viability was assessed by measuring Alamar Blue incorporation according to the manufacturer's protocol (Invitrogen). Percent growth inhibition was calculated relative to the control DMSO wells.

### Secondary Screen/Dose Response Curve

The potency and selectivity of hits from the primary screen was tested in 8-point two-fold dilution series ranging from 50 μM-0.39 μM concentrations with more lines of GNS, NS and fibroblast. Experimental conditions were the same as in primary screen. IC₅₀ was calculated based on an approximate observed value. Fold selectivity was calculated as IC₅₀ of BJ/IC₅₀ of any GNS cells with lowest IC₅₀.

### Patient Derived Xenografts

All mouse procedures were approved by the Hospital for Sick Children's Animal Care Committee. To validate the in vivo effect of L-741,742 and PNU 96415E, 2×10⁵ GNS cells in 200 μl of PBS and matrigel (1:1) were injected subcutaneously into flanks of non-obese diabetic/severe combined immunodeficient (NOD/SCID) female mice. 8 mice (2 tumours per mouse except for one mouse in control group that has one tumour) were maintained for each group; control (15 tumours), L-741,742 (16 tumours) and PNU 96415E (16 tumours). L-741,742 and PNU 96415E were dissolved in 40% 2 hydroxy β-cyclodextrin (Sigma). Mice were treated three days after tumour implantation. Both L-741,742 and PNU 96415E were injected (20 mg/kg) i.p for 5 days on two days off until the end point. Control group was injected with 40% 2 hydroxy β-cyclodextrin. Tumour growth was monitored with microcalipers until tumour volume reached 17 mm in any one tumour from any group and all mice were sacrificed at the same end point. Dissected tumour volume was measured and mass was determined by weighing.

cAMP Assay

cAMP levels were measured with an ELISA-based cAMP assay kit purchased from Cell Signaling (#4439). GNS (G362) cells were seeded overnight in a 96 well plate and treated with forskolin (30 μM) for 15 minutes, or pretreated with DRD4 agonist A412997 (30 μM) for 15 minutes followed by forskolin treatment. Cells were lysed and processed as per manufacturer's protocol.

### Statistical Analysis

All grouped data are presented as mean±SEM unless otherwise stated in figure legends. Statistical significance difference between groups was assessed by Student's I-test,

### Accession Number

The GenBank accession number for the PNU 96415E treated GNS microarray data described in this manuscript is GSE62714.

### Gene Expression Profiling

GNS cells (G362 and G411) were treated with PNU 96415E (25 μM) for 0 h (Control), 24 h and 48 h, and cells were lysed for RNA at each time point using RNeasy kit (Qiagen). RNA extracted from the samples was hybridized on Affymetrix Human Gene 1.0 ST arrays using standard protocol (TCAG, Toronto, Ontario, Canada). RMA background correction, quantile normalization and log 2 transformation were applied to the CEL files using the Bioconductor affy package (R 3.0.1, affy package version 1.38.1). Batch correction was applied using ComBat function from sva (3.6.0) and gene annotations were retrieved using hugene10sttranscriptcluster.db (8.0.1). Genes were ranked based on the average log fold change (log FC) of the 2 treated GNS (G411 and G362) at 24 h or 48 h to vehicle (0 h) samples. The data were analyzed using GSEA (Subramanian et al., Proc. Natl. Acad. Sci. 2005. 102(43), 15545-15550) with parameters set to 2000 gene-set permutations and gene-sets size between 8 and 500. The gene-sets included in the GSEA analyses were obtained from KEGG, MsigDB-c2, NCI, Biocarta, JOB, Netpath, HumanCyc, Reactome and the Gene Ontology (GO) databases, updated Oct. 14, 2013 (http://baderlab.org/GeneSets). An enrichment map (version 1.2 of Enrichment Map software (Merico et al., 2010. PLoS One 5(11), e13984) was generated for each comparison using enriched gene-sets with a False Discovery Rate<0.02% and the overlap coefficient set to 0.5.

### In Vitro Limiting Dilution Assay (LDA)

Limiting dilution assay was performed as described previously (Tropepe et al., 1999. Developmental Biology 208, 166-188). Primary tumours were dissociated into single cell suspension and seeded into a 96-well plate with 10 point-2 fold serial dilution starting from 2000 cells to 4 cells/well, 6 wells for each dilution per plate. Each well was scored for neurosphere formation after 14 days of incubation. Percent of wells not containing spheres for each cell density was calculated and plotted against the cells per well, regression lines were plotted and x-intercept value at 0.37 was calculated at 95% confidence interval using Sigma Plot, which gives the number of cells required to form at least one neurosphere.

### Western Blots

Western blots were performed using the following antibodies; anti-DRD4 antibody at 1:750 (Millipore# MABN125), anti-activated MAPK at 1:2500 (Promega#V803A), anti-ERK1/2 at 1:2000 (Promega#V114A), anti-βactin at 1:10,000 (Sigma), anti-LC3B at 1:1000 (Cell Signaling #3868), anti-p62 at 1:1000 (BD Bioscience), anti-LAMP1 at 1:2000 (Developmental Studies Hybridoma Bank), anti-mono and polyubiquitinylated protein conjugates (FK2) at 1:1000 (Enzo Life Sciences).

### Short Hairpin Construct and Transfections

5 μg of short hairpin targeting DRD4 (RHS4533-EG1815; TRCN0000014453; Thermo Scientific) or control shRNA construct targeting eGFP (RHS4459; Thermo Scientific) were transfected in 1×10⁶ GNS cells using the Amaxa Nucleofector kit (VPG-1004) and Nucleofector II electroporator (Amaxa Biosystem) according to manufacturer's protocol. After 24 h transfection, cells were briefly selected with puromycin for 48 h and seeded for proliferation assay without selection. Two wells from each transfection were maintained after electroporation; one well was lysed to check for knockdown by western blot and the other well was seeded for proliferation assay.

### Transmission Electron Microscopy

Analysis was performed at the Bioimaging facility at Mt Sinai Hospital, Toronto. Cells were harvested, pelleted and fixed in 2% glutaraldehyde in 0.1M sodium cacodylate buffer, rinsed in buffer, post-fixed in 1% osmium tetroxide buffer, dehydrated in a graded ethanol series followed by propylene oxide, and embedded in EMBed 812 resin. Sections 100 nm thick were cut on an RMC MT6000 ultramicrotome, stained with uranyl acetate and lead citrate and viewed in an FEI Tecnai 20 TEM.

### Phospho-Kinase Array

A human phospho-kinase antibody array was purchased from R&D systems (Cat# ARY003). This array contains capture antibodies for 43 kinases in duplicate on nitrocellulose membrane. GNS (G362) and NS (hf5205) lines were treated with L-741,742 (10 μM) and PNU 96415E (25 μM) along with a DMSO control for 24 h, and cells were then processed according to the manufacturer's protocol. Signal intensity was quantified using ImageJ.

### Combination/Synergy Screen

2000 GNS cells (G362 and G481) were seeded in a 96 well plate and treated with a combination of 6-point 2-fold dose series of either L-741,742 (6.25 μM-0.39 μM) or PNU 96415E (25 μM-1.56 μM) with 10-point 2-fold dose series of temozolomide (100 μM-0.39 μM) in 60 point combination doses. The cells were incubated with a combination of the two drugs for five days and then checked for cell viability using the alamar blue assay. Combination index (CI) plot and CI value was calculated for 5 point dose series in each combination using the programme COMPUSYN. Data points taken for COMPUSYN analysis are temozolomide (100, 50, 25, 12.5 and 6.25 μM) in combination with either L-741,742 (6.25, 3.12, 1.56, 0.78 and 0.39 μM) or PNU 96415E (25, 12.5, 6.25, 3.12 and 1.56 μM).

### Results:

### Identification of GNS-Selective Compounds.

To identify compounds that selectively inhibit the growth of GBM-derived neural stem cells (GNS), proliferation assays were established for three different cell types: GNS cells, normal human fetal neural stem cells (NS) and the BJ human fibroblast cell line. GNS cells were patient-derived tumour cells established and maintained as an adherent monolayer in serum-free medium with epidermal growth factor (EGF) and basic fibroblast growth factor (FGF); these cell lines retain tumour-initiating potential and regeneration of tumour cellular hierarchies when implanted into immunocompromised mice. GNS cells display many characteristics of normal NS cells including expression of the markers, Nestin and SOX2, and the ability to self-renew and to partially differentiate. Thus, NS cells serve as a well-matched control for their neoplastic GNS counterparts. To eliminate compounds with non-specific cytotoxic effects, NS-selective hits were defined as those that target both NS and GNS cells, but not fibroblasts. Compounds were then filtered for those that showed more activity towards GNS cells compared to NS cells, and these were termed ‘GNS-selective’ compounds.

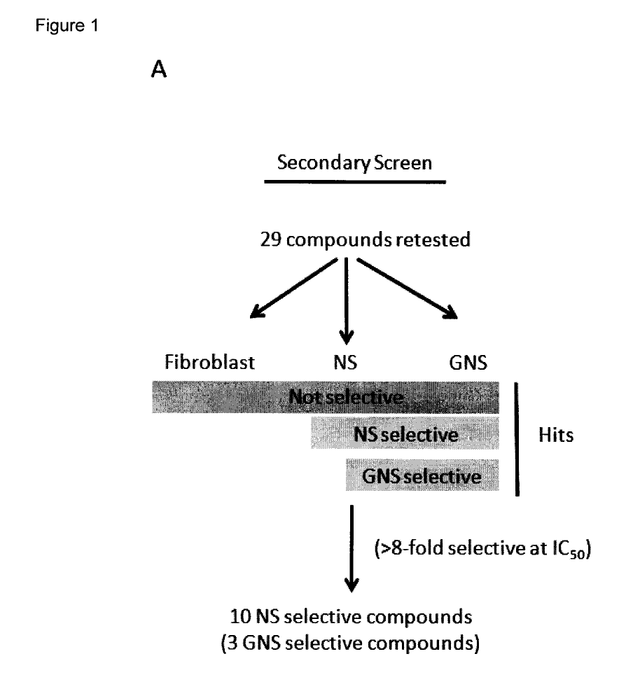

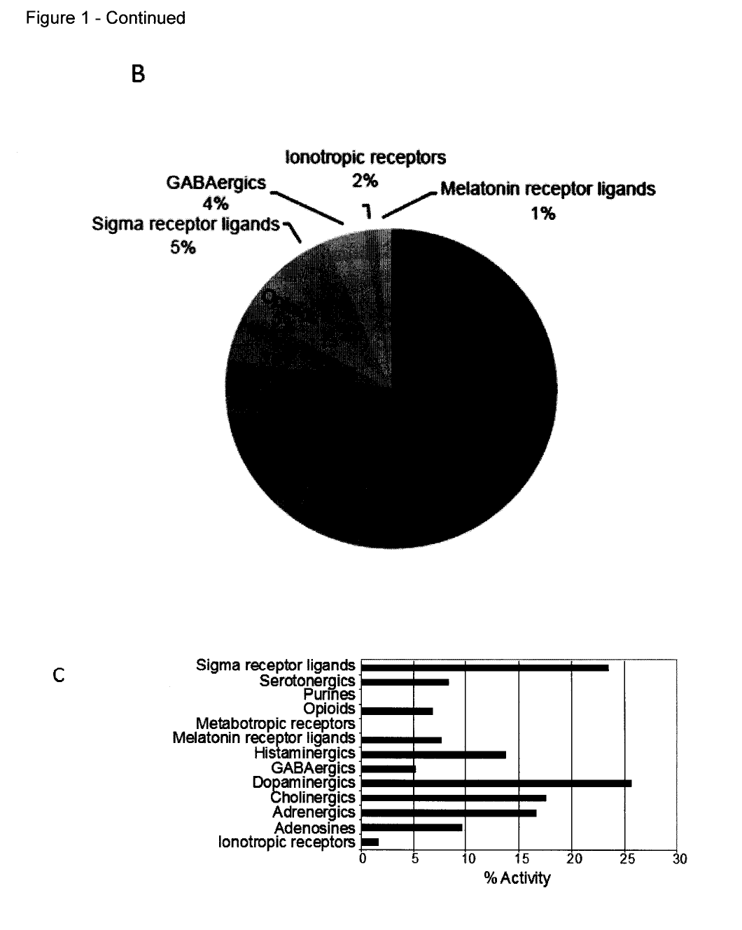

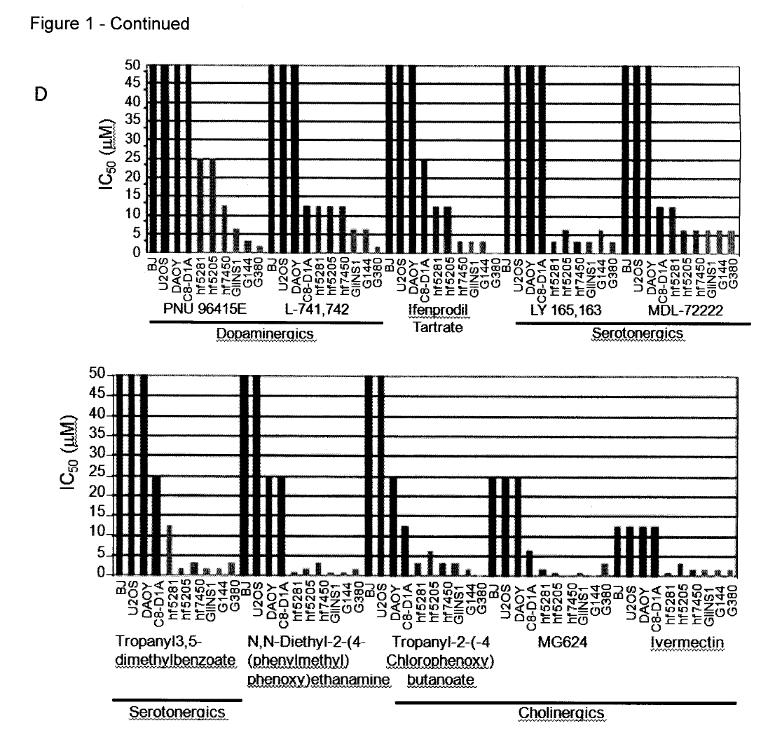

A BIOMOL library of 680 neuroactive compounds were screened against three GNS lines (GliNS1, G179 and G144), two NS lines (hf5205, hf5281) and the BJ fibroblast line at a concentration of 5 μM for five days (FIG. 1A). Primary hits were defined as compounds that caused greater than 20% growth inhibition compared to the DMSO control. The total hit rate in all cell populations ranged from 2.6-6.5% (FIG. 1A). Of all the neurochemical classes, compounds known to modulate dopaminergic (27%), cholinergic (17%), adrenergic (18%) and serotonergic (9%) pathways were enriched in the total hits, suggesting that these pathways may play a specific role in regulating NS cell growth (FIG. 1B). These pathways were also the main enriched hits when normalized to each neurochemical class, defined as the number of hits per number of compounds in each class (FIG. 1C).

Twenty nine compounds that showed a selective effect on GNS and NS cells compared to fibroblasts were selected for further study. The 29 compounds were retested in a dose response series (0.39-50 μM) in the same cell populations as in the primary screen. From this secondary screen, ten compounds were selected that showed more than 8-fold selectivity towards GNS and NS cells compared to fibroblasts. Fold selectivity was calculated as IC₅₀ of BJ/IC₅₀ of any of the NS or GNS lines that showed the lowest IC₅₀. These ten NS-selective compounds were PNU-96415E, L-741,742, Ifenprodil tartrate, LY-165,163, MDL-72222, Tropanly 3,5-dimethylbenzoate, N,N-Diethyl-2-(4-(phenylmethyl)phenoxy)ethanamine, (±)-Tropanyl-2-(4-chlorophenoxy)butanoate, MG-624 and Ivermectin. One compound, cis-(±)-N-Methyl-N-[2-(3,4-dichlorophenyl)ethyl]-2-(1-pyrrolidinyl)cyclohexamine 2HBr that showed 8-fold selectivity was not available for further study. The ten NS-selective compounds were enriched for dopaminergic, serotonergic and cholinergic classes (FIG. 1D) suggesting these pathways as potential targets for GBM. To further validate selectivity, each compound was tested in three further non-NS control cell lines, namely Daoy cells (a human medulloblastoma cell line), U-2 0S (a human osteosarcoma cell line) and C8-D1A (a mouse astrocyte line). The ten compounds were 8-128 fold more active against NS or GNS cells compared to BJ fibroblasts. Notably, three of the compounds PNU 96415E, L-741,742 and ifenprodil tartrate, showed 8-fold selectivity against GNS cells compared to NS cells and were termed GNS-selective compounds. Two of these compounds, PNU 96145E and L-741,742, represent DRD4 antagonists and were chosen for further investigation.

### DRD4 Antagonists Inhibit GNS Growth and Reduce Clonogenic Cell Frequency in Primary GBM Tumour Cells.

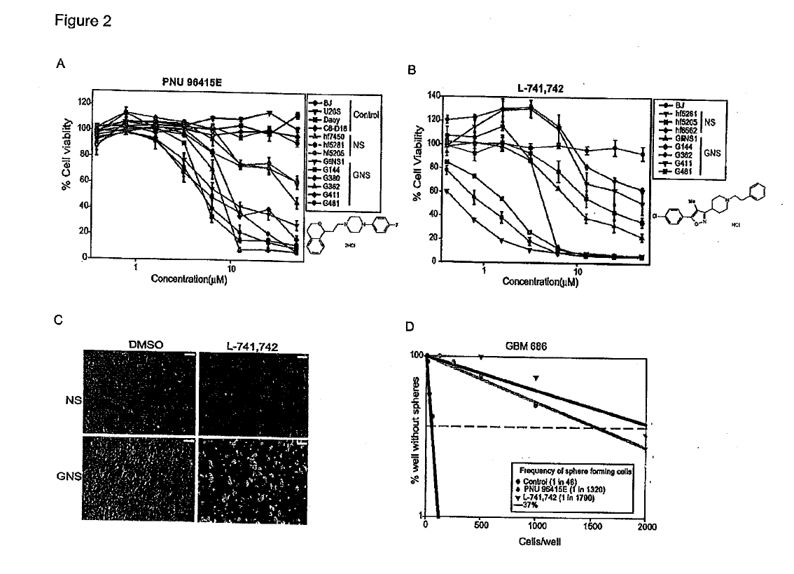

PNU 96145E and L-741,742 were retested alongside a panel of other commercially available DRD4 antagonists (L-745,870 and PD 168568) to determine whether they showed a similar effect on growth inhibition of GNS cells. When tested against six GNS and four NS lines, all DRD4 antagonists showed selectivity toward ONS cells with differing potency (IC₅₀), in the order of L-741,742 (1.5-6.2 μM)>L-745,870 (3.1-6.2 μM)>PNU 96415E (6.25 μM)>PD168568 (25-50 μM). L-741,742 and PNU 96415E are specific DRD4 antagonists and showed the greatest selectivity towards GNS cells (FIG. 2A-C). PNU 96415E displayed robust selectivity towards GNS cells compared to NS cells and non-NS control cells, the latter of which were not sensitive even at the highest concentration tested (50 μM) (FIG. 2A). L-745,870 was also a potent GNS selective inhibitor, while PD 168568 showed a selective effect at a much higher concentration of around 25-50 μM.

To confirm that the effect of DRD4 antagonism was not merely specific to GNS cell lines, L-741,742 and PNU 96415E were tested in freshly isolated primary GBM patient tumour cells (n=3) using a primary in vitro limiting dilution assay (FIG. 2D). Tumour samples were dissociated into a single cell suspension and directly seeded prior to treatment with L-741,742 (10 μM). PNU 96415E (25 μM) or DMSO control for 14 days before scoring wells for presence/absence of neurosphere colonies. A massive reduction in frequency of colony forming cells after treatment with L-741,742 (40-83 fold reduction) and PNU 96415E (19-29 fold reduction) was observed in comparison to the control (FIG. 2D). These data strongly suggest that both L-741,742 and PNU 96415E inhibit the clonogenic potential of fresh primary tumour cells, and may therefore effectively target the stem cell population in each patient tumour.

### DRD4 Antagonists Inhibit GEM Xenograft Growth In Vivo

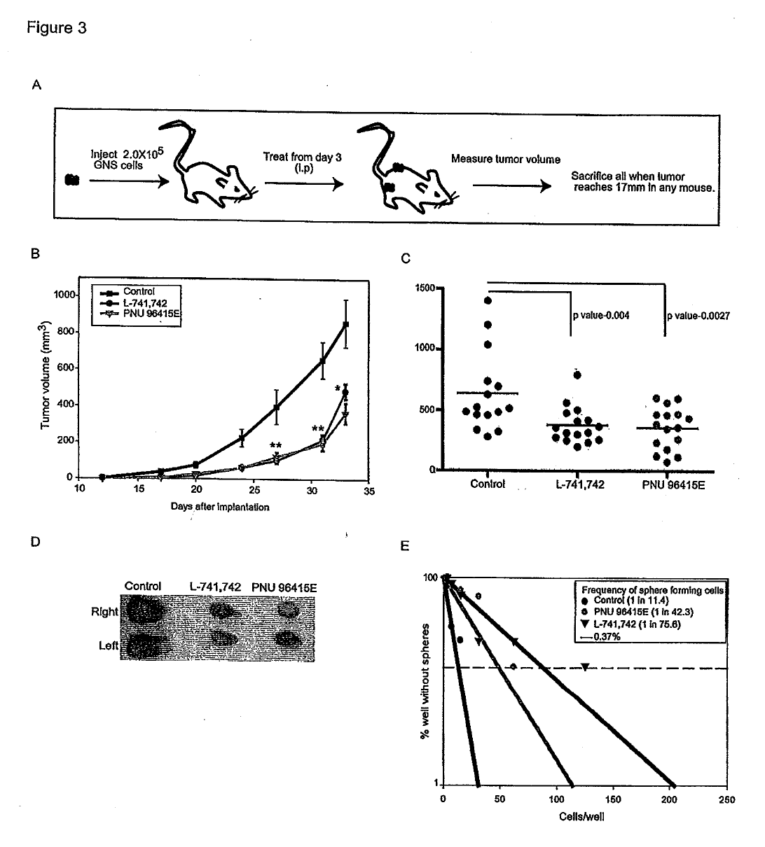

To test the effects of L-741,742 and PNU 96415E in vivo, GNS cells were subcutaneously injected into the flanks of immuno-compromised NOD scid gamma (NSG) mice followed by treatment with an intraperitoneal injection of PNU 96415E (20 mg/kg), L-741,742 (20 mg/kg), or vehicle, with a dosing regimen of five days on and two days off until tumours reached the institutional volumetric cutoff of 17 mm in any one mouse (FIG. 3A). The effect of PNU 96415E and L-741,742 treatments were tested by three different measures. Measurement of tumour volume over the course of the four week time course revealed much slower growth in the treated groups compared to the vehicle control group (FIG. 3B). The average tumour weight at the end point was reduced by 44.3% with PNU-96415E treatment (p value=0.0027) and 40.9% with L-741,742 treatment (p value=0.004) (FIG. 3C-D). Control and treated tumours were then dissociated and subjected to primary in vitro limiting dilution assays to determine if L-741,742 and PNU-96415E affected the clonogenic capacity of the in vivo treated tumours. A substantial reduction in frequency of colony forming cells in both the treated groups compared to control (FIG. 3E) was observed, indicating a reduction in the stem cell fraction of the treated tumours. The colony forming cell frequency was reduced in treated groups by 4-7 fold, from 1 in every 11 cells in the vehicle treated tumours to 1 in every 43 cells in PNU 96415E treated tumours or 1 in every 76 cells in L-741,742 treated tumours (FIG. 3E). The effect of PNU 96415E in vivo was further validated using an independent GNS cell line (G411), and a similar reduction in tumour growth rate and end-point size was observed. Together, these data in human patient-derived xenograft models demonstrate the clinical potential for DRD4 antagonism in treating GBM.

### Primary GBM Tumour and GNS Cells Express Functional DRD4 Receptor

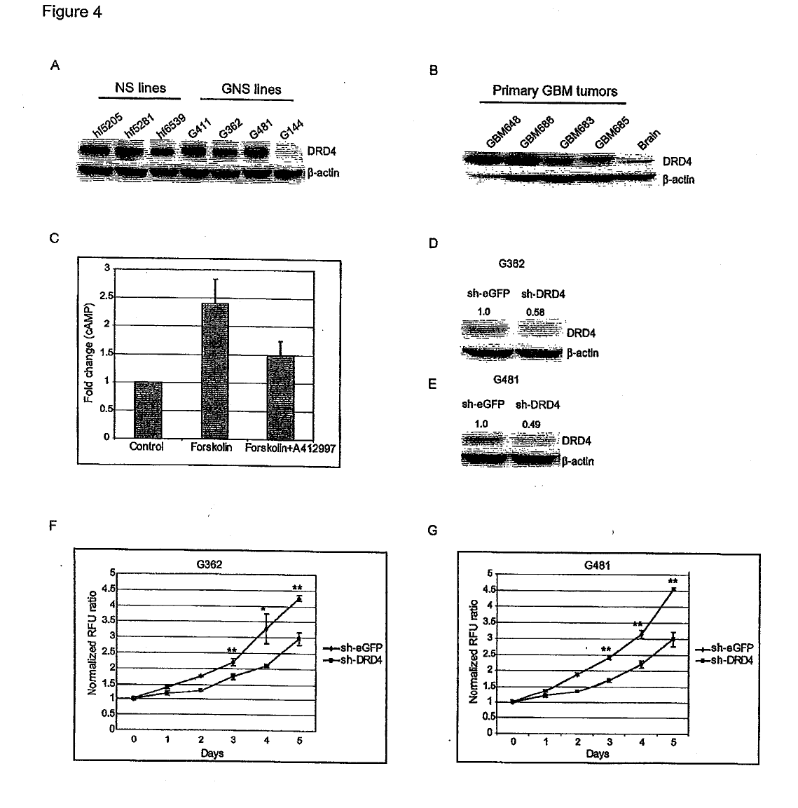

To determine if DRD4 antagonists exert their effects directly through the DRD4 receptor, it was first confirmed that DRD4 was expressed in both GNS and NS cells by western blot (FIG. 4A). Interestingly, primary GBM tissue samples expressed DRD4 at a higher level than normal brain tissue (FIG. 4B). To assess DRD4 function in GNS cells, a known downstream readout of receptor activity was determined. DRD4 is a dopamine D2-like receptor that inhibits adenylate cyclase and decreases cAMP levels. GNS cells were treated with forskolin to activate adenylate cyclase and increase cAMP, and then it was assessed whether activation of the DRD4 receptor by the DRD4-specific agonist A412997 could block the forskolin-induced cAMP response. Forskolin treatment in GNS cells increased cAMP concentration by 2.4 fold and pretreatment with DRD4 agonist A412997 blocked this response by 38%, confirming that DRD4 functions as expected in GNS cells (FIG. 4C). Primary tumour and tumour-derived GNS cells thus express DRD4 and can respond to DRD4-dependent signals.

### Knockdown of DRD4 Suppresses GNS Growth

To validate DRD4 as a therapeutic target in GBM and determine if loss of its function phenocopies the effect of PNU 96415E and L-741,742, shRNA-mediated knockdown experiments were performed and the effect on cell proliferation was measured. Five lentiviral shRNA constructs from The RNAi Consortium (TRC) against human DRD4 were tested using shRNA-eGFP as a positive control. Only one out of five shRNA-DRD4 constructs (shRNA-DRD4-4: TTGAGGCCGCACAGTACGGGC (SEQ ID NO: 3)) caused consistent knockdown at 72 hours post transfection. Reduced DRD4 expression after transduction of the shRNA-DRD4 construct was confirmed in two separate GNS lines (FIG. 4D-E). This knockdown was accompanied by a significant reduction in proliferation compared to control shRNA-transfected cells (FIG. 4F-G). These results confirm the inferred role for DRD4 function in GNS cell growth.

### Effect of DRD4 Antagonism on Gene Expression Patterns

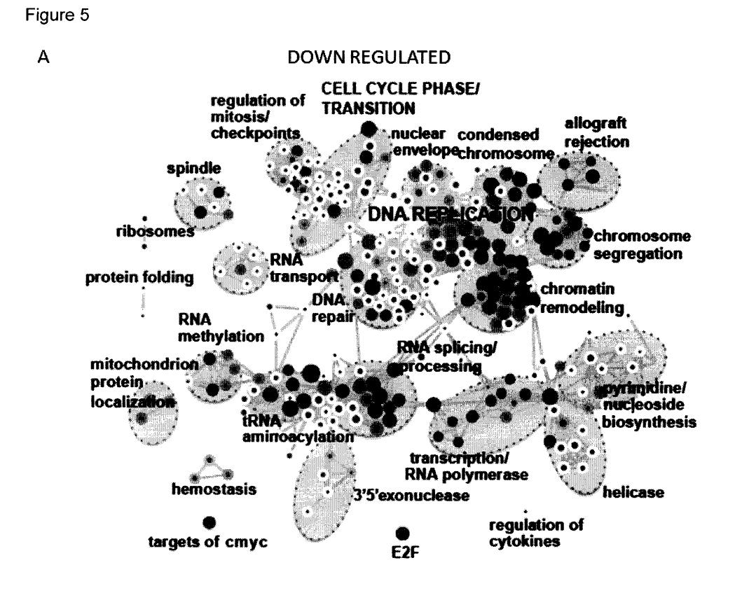

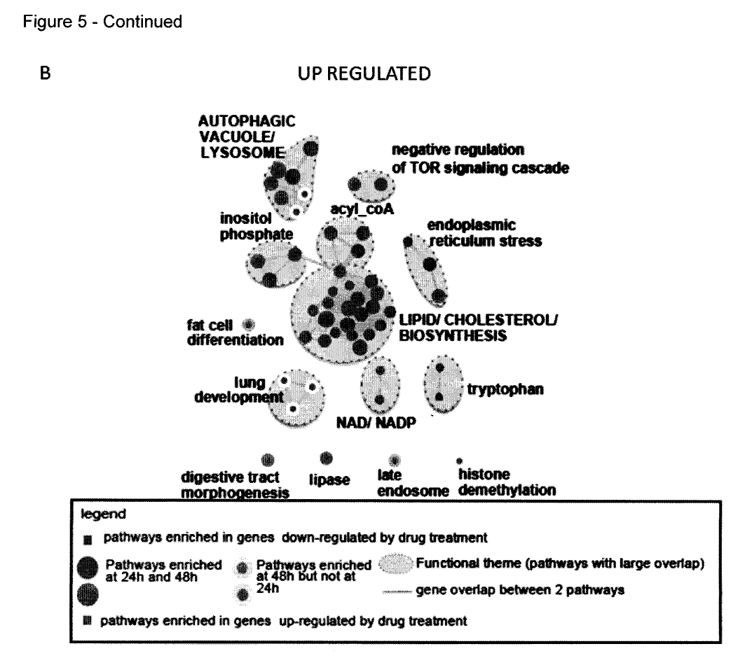

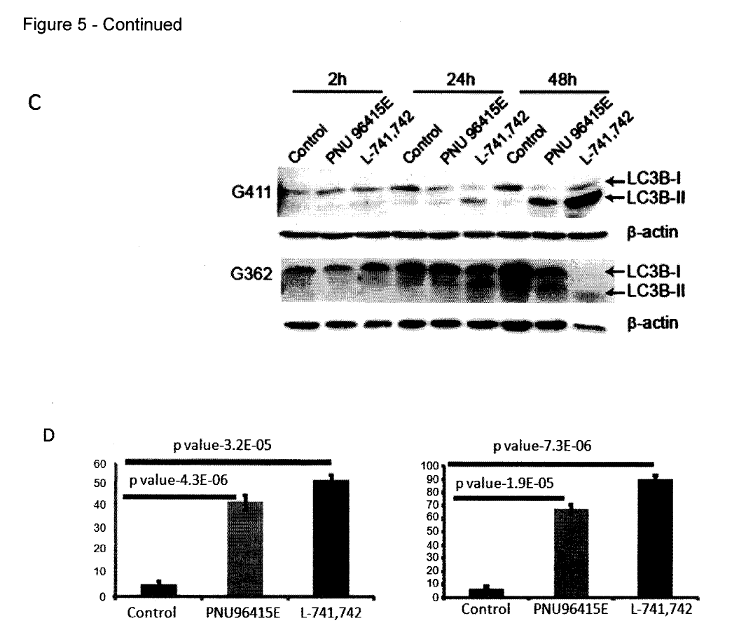

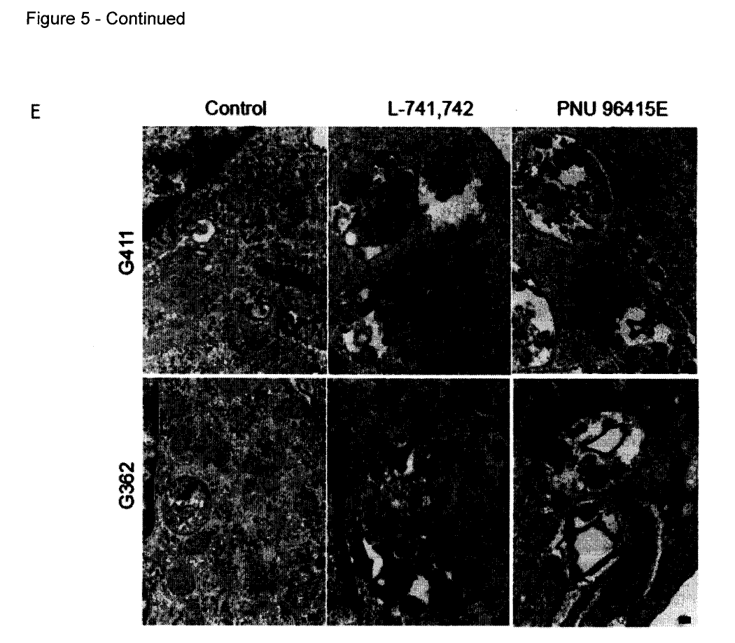

The mechanism of action for a DRD4 antagonist was then characterized through global gene expression profiles. Two GNS lines (G362 and G411) were treated with PNU 96415E (25 μM) for 24 h and 48 h and analyzed for differential effects of PNU 96415E on gene expression by microarray analysis. Gene set enrichment analysis (GSEA) was used to identify pathways enriched in differentially regulated genes upon PNU 96415E treatment. Genes were ranked based on the average log-fold change of PNU96415E treated GNS cells at 2411 or 48 h compared to control. Gene-sets (pathways) with a false discovery rate (FDR) equal to or less than 0.2% (0.002) were considered significantly altered upon PNU 96415E treatment (FIG. 5A-B). Genes that were down regulated at 48 h were enriched in 172 gene sets that are highly connected, as categorized into 25 main biological functions including DNA replication, chromatin remodeling, DNA repair, cell cycle, and RNA splicing (FIG. 5A). Overlap of the top down-regulated genes (fold change<−1.5) in both G362 and G411 cell lines revealed genes involved in DNA replication (MCM10, EXO1, CDC45, ORC1) and cell cycle phase transitions (CDC25A, CCNE2). For genes up-regulated upon PNU 96415E treatment, enrichment was observed in 45 gene sets (FDR=<0.002) that comprised 14 main pathways including lipid/cholesterol biosynthesis, autophagic vacuoles and lysosomes (FIG. 5B). Overlap of the top up-regulated genes (fold change>1.5) uncovered pathways involved in cholesterol biosynthesis (TM7SF2, DHCR7, MVD, HSD17B7) after 24 h and autophagic vacuole formation (TP53INP1, GABARAPL1, WIPI1, SQSTM1) after 48 h. This expression analysis suggested that the pathways involved in DNA replication and cell cycle progression were inhibited by DRD4 antagonism, while pathways in lipid metabolism and autophagy were activated.

### DRD4 Antagonism Causes Massive Accumulation of Autophagic Vacuoles

Prompted by the pronounced up-regulation of autophagy genes in response to DRD4 inhibition, autophagy status in GNS cells was assessed using the autophagy marker LC3-II. When autophagy is induced, the cytoplasmic form of LC3-I (microtubule associated protein 1 light chain3-I) is conjugated to phosphatidylethanolamine (PE) to form LC3-II, which then translocates to the autophagosome membrane. This conversion of LC3-I to LC3-II serves as a hallmark for autophagosome formation and can be measured as a molecular mass shift in western blots and as LC3⁺ puncta by immunocytochemical staining. L-741,742 (10 μM) and PNU 96415E (25 μM) treatment in GNS cells (G411 and G362) caused an increase in levels of LC3B-II consistent with accumulation of autophagosomes (FIGS. 5C & 6B). Increased LC3B⁺ puncta in GNS cells upon treatment was also observed, with more than 50% cells showing large LC3B⁺ puncta after 4811, indicating the presence of autophagosomes (FIG. 5D). Accumulation of autophagosomes was further corroborated by transmission electron microscopy. L-741,742 and PNU 96415E treatment in both G411 and G362 caused the formation of large autophagic vacuoles containing various cellular fragments, accompanied by double membrane autophagosomes and autolysosomes. (FIG. 5E). Together, these experiments revealed a massive accumulation of autophagic vacuoles in GNS cells after antagonism of the DRD4 receptor.

### Autophagosome Accumulation is Due to a Block in Autophagic Flux

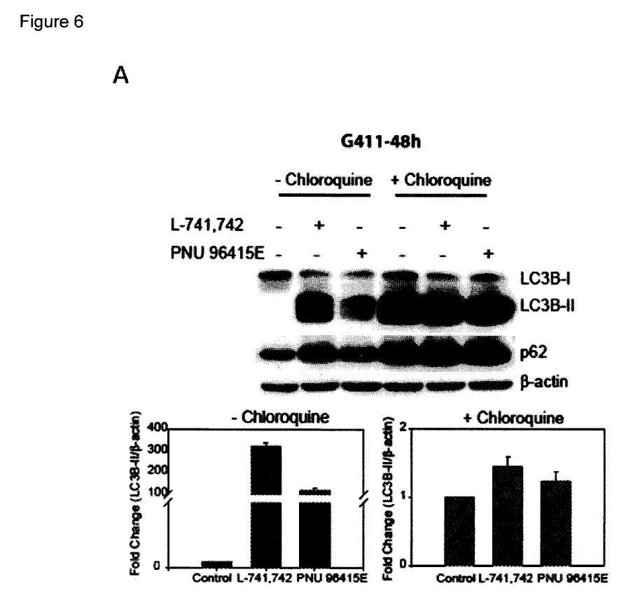

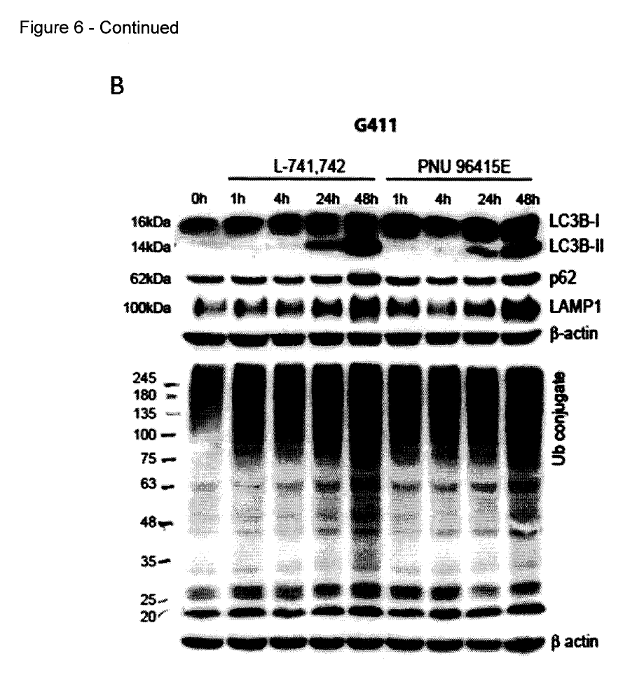

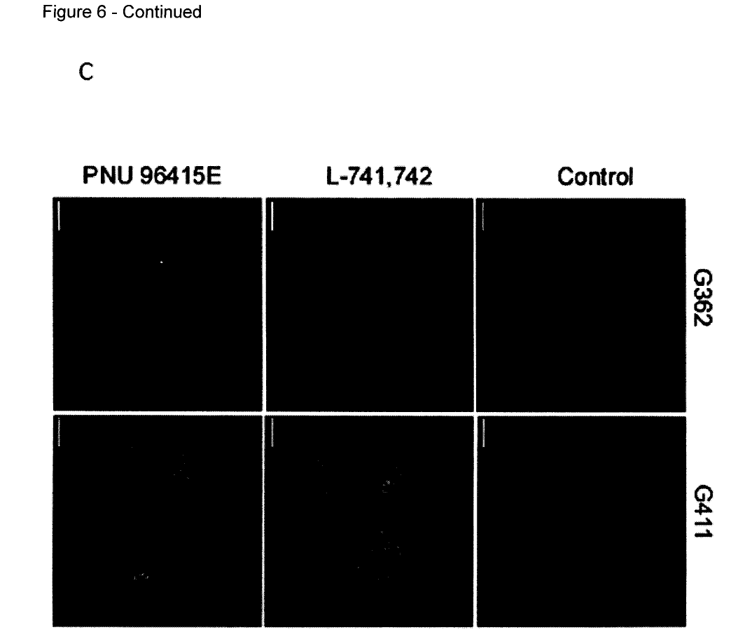

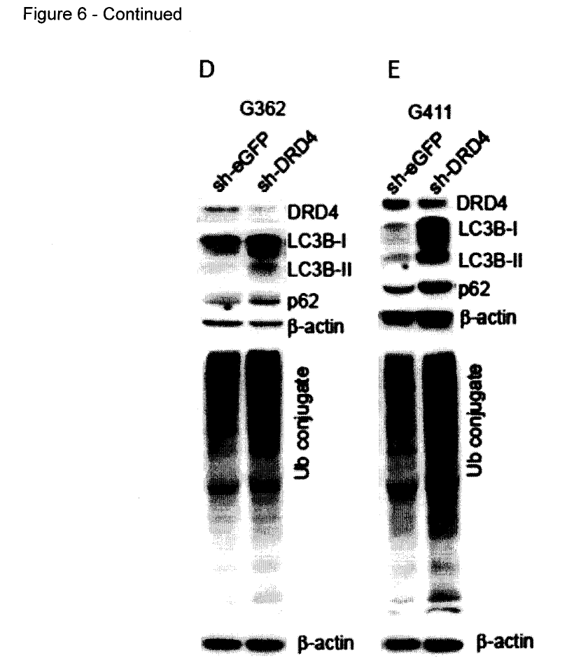

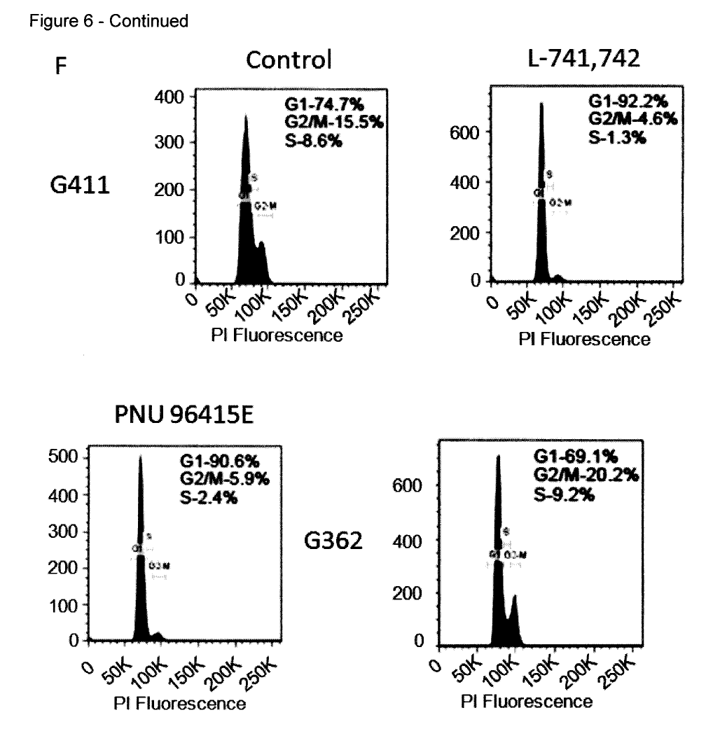

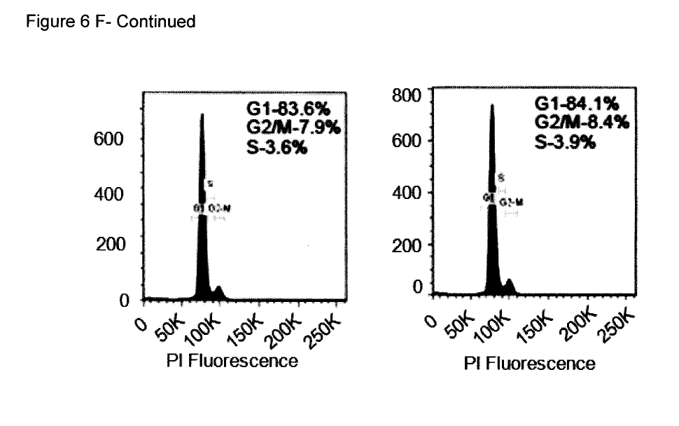

An increase in LC3-II levels, and autophagosome number, can result from either the induction of autophagy or the inhibition of autophagic flux at a late stage. Autophagic flux is defined as the complete process of autophagy from the formation of phagophore to the fusion of autophagosome with lysosomes and subsequent degradation of autophagic cargo. This flux can be measured by assessing LC3-II turnover in the presence or absence of inhibitors of lysosomal degradation such as chloroquine, which prevents acidification of lysosomes and subsequent degradation of autolysosome contents. In chloroquine treated cells, an autophagy inducer will increase LC3-II levels, where as an autophagy blocker will not change LC3-II levels. In the presence of chloroquine, L-741,742 and PNU 96415E treatment did not increase LC3-II levels compared to control, despite the fact both drugs increased LC3-II levels when administered alone (FIG. 6A). These data demonstrate that the effect of DRD4 antagonism on LC3-II levels is a result of blocked autophagosome degradation.

Clearance of the autophagy-specific substrate p62, which reflects autophagy turnover, was then assessed. As predicted for a block in autophagic flux, p62 accumulated along with the increase in LC3B-II in L-741,742 or PNU 96415E treated GNS cells (FIG. 6B). Consistent with impairment in autophagy, an increase in undegraded ubiquitinated protein conjugates in treated cells was observed (FIG. 6B). An increased level of LAMP 1 (lysosome associated membrane protein 1) and LysoID positive puncta was also observed, both of which indicate an increase in lysosomes due to impaired autophagic flux (FIG. 6B). Co-localization of autophagosomes with lysosomes was then assessed using the autophagosomal probe CytoID-Green and the lysosomal probe LysoID-Red in live GNS cells treated with DRD4 antagonists. It was noted that the autophagosomal marker did not colocalize with the lysosomal marker, consistent with a block in the fusion of autophagosomes with lysosomes (FIG. 6C). These data show that the observed accumulation of LC3-II levels upon DRD4 antagonism was not due to autophagy induction but rather to a block at a late stage of autophagy that results in massive accumulation of enlarged autophagic vacuoles.

To confirm that the impairment of the autophagy/lysosomal degradation pathway induced by L-741,742 and PNU 96415E was due to inhibition of DRD4, autophagic flux was assessed after shRNA knockdown of DRD4 in GNS cells. Increased levels of LC3-II in DRD4 knockdown cells was observed compared to sh-eGFP transduced controls (FIG. 6D-E). This increase in LC3-II was accompanied by accumulation of p62, LAMP1 and ubiquitinated protein conjugates, all consistent with a block in autophagic flux (FIG. 6D-E). These data demonstrate that the DRD4 antagonists exert their effects on autophagy in GNS cells via DRD4.

DRD4 Antagonists Trigger a G₀/G₁ Phase Arrest

As DRD4 antagonists inhibit proliferation of GNS cells accompanied by decreased expression of DNA replication and cell cycle genes, the effect of DRD4 antagonists on the cell cycle was then assessed. Flow cytometric analysis of DNA content in G411 and G362 cells treated with either L-741,742 or PNU 96415E revealed a G₀/G₁ arrest and a reduction S phase and G₂/M phase cells in a time dependent manner (FIG. 6F). The viability of GNS cells progressively decreased from 1 to 5 days of treatment as judged by increased trypan blue staining and an inability to regrow after replating in fresh medium. As no increase in caspase 3/7 activity or cleaved PARP in the treated cells was observed, DRD4 appears to cause G₀/G₁ arrest and subsequent non-apoptotic cell death.

### Inhibition of ERK1/2 Signaling by DRD4 Antagonism

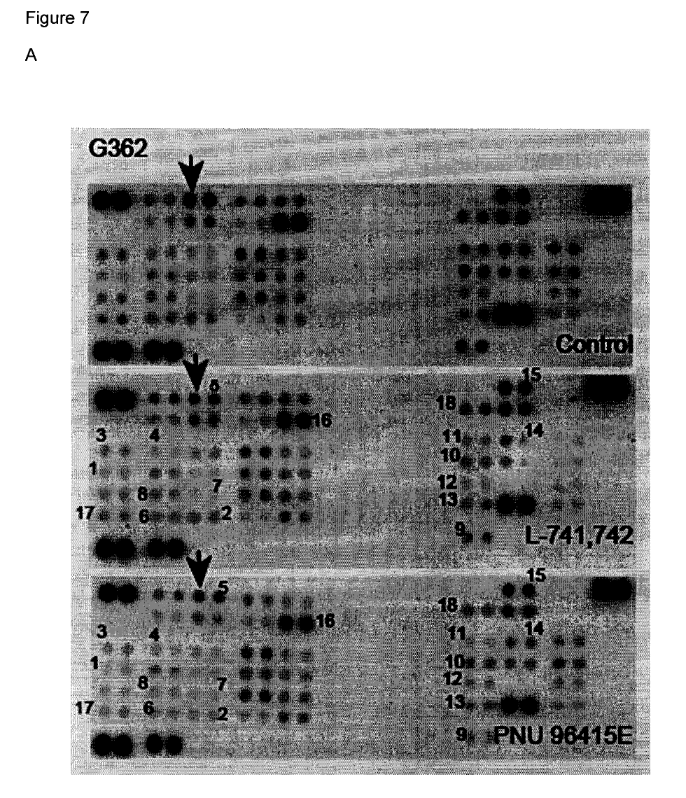

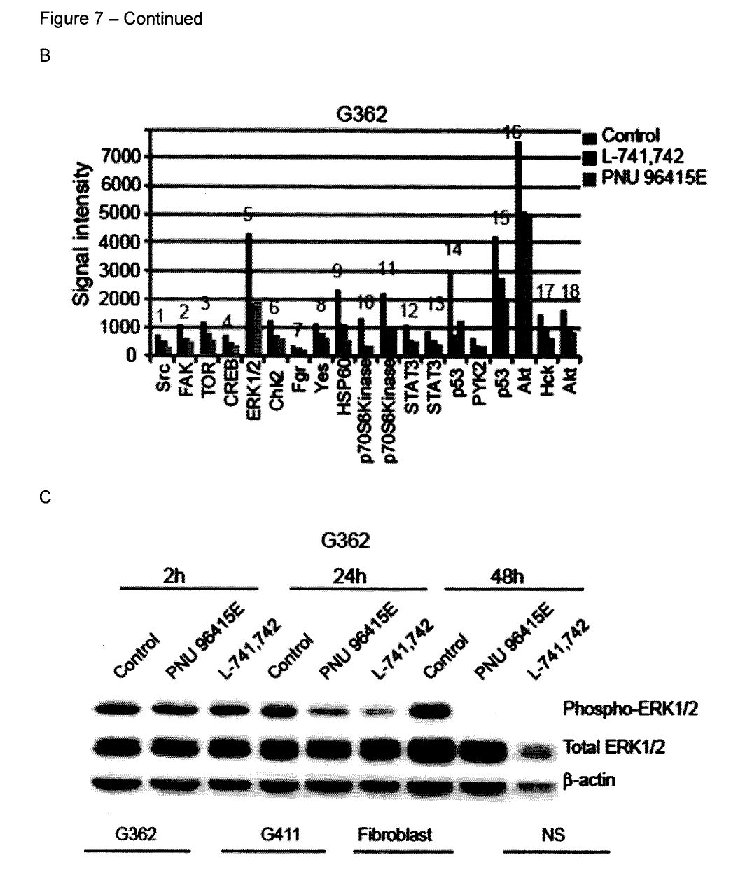

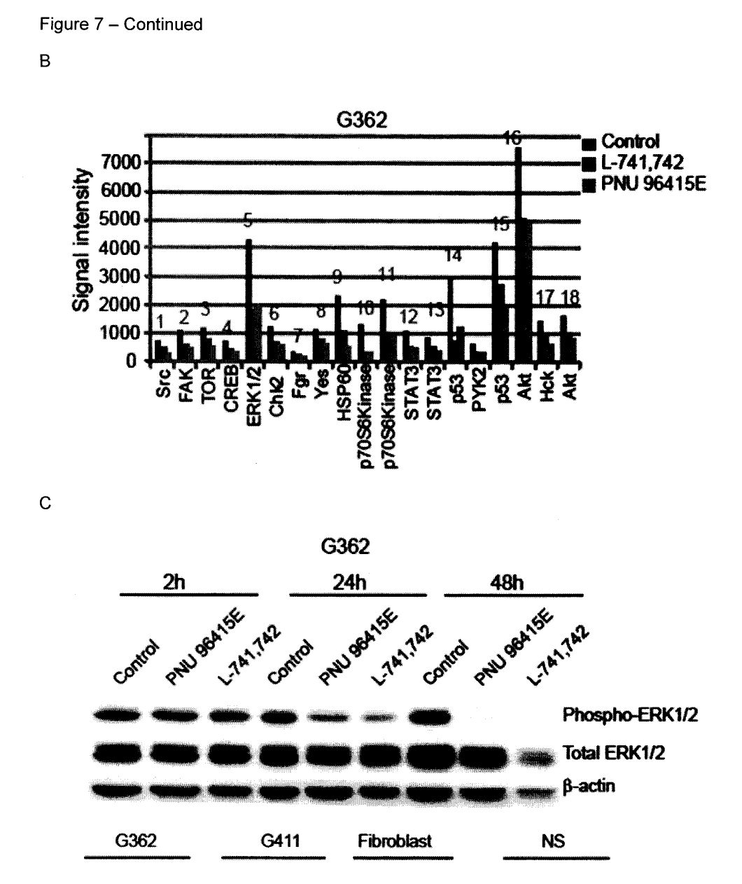

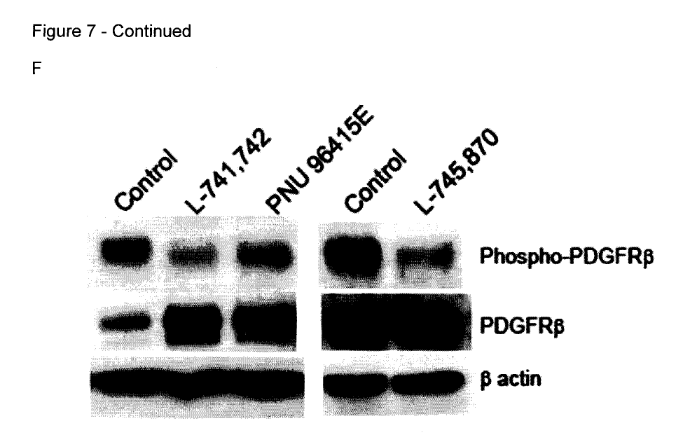

To determine how DRD4 receptor antagonism in GNS cells may mediate growth inhibition, the phosphorylation status of 43 kinases and substrates implicated in various signaling pathways in GNS cells versus NS cells was determined using a dot blot assay. Cells were treated with L-741,742 (10 μM) and PNU 96145E (25 μM) for a period of 24 h and protein lysates were harvested and assessed with a phosphoprotein antibody array (FIG. 7A). Eighteen (18) phosphoproteins were found to exhibit a decrease in phosphorylation upon treatment in GNS cells (FIG. 7B). These kinases and substrates included ERK1/2, p70S6 kinase, HSP60, p53, FAK, STAT3, CREB, AKT and TOR, all of which are key signaling molecules that regulate cell growth and division. ERK1/2 was one of the top hits in the array, with a 40% reduction compared to the untreated control. DRD4 is known to activate ERK1/2 by transactivation of platelet derived growth factor receptor β. The selectivity of DRD4 antagonism to GNS cells was reflected in the much more modest changes in phospho-profile of NS cells after treatment with both compounds.

The effect of DRD4 antagonism on ERK1/2 phosphorylation in GNS and NS cells was validated by western blot at various time intervals and a decrease in ERK1/2 phosphorylation over time in GNS cells but not in NS cells was observed (FIG. 7C-D). It was also confirmed that transient DRD4 knockdown decreased ERK1/2 phosphorylation in GNS cells compared to control shRNA-eGFP transfected cells (FIG. 7E-F). These biochemical data suggest that the DRD4 antagonists act on target and that DRD4 regulates GNS cell growth in part through the central ERK1/2 pathway.

DRD4 Antagonists are Synergistic with TMZ

The effect of DRD4 antagonists in conjunction with the conventional chemotherapeutic agent, temozolomide (TMZ) was evaluated to assess the clinical potential of this drug pair combination. Synergy in both G362 and G481 cells using the combination of TMZ with either L-741,742 or PNU 96415E was assessed. Both L-741,742 and PNU 9641E exhibited striking synergism with TMZ in vitro (FIG. 8 A-D). The degree of synergism was quantified using the combination index (CI) method (Chou, Cancer Research. 2010. 70, 440-446) for which a CI value of 1 indicates additivity, a value of <1 indicates synergism and a value >1 indicates antagonism. The lowest CI value for L-741,742 in combination with TMZ in G481 and G362 was 0.28 and 0.29 respectively, and for PNU 96415E in combination with TMZ was 0.32 and 0.56 respectively (FIG. 8E-F). Based on these in vitro data, both DRD4 antagonists enhance the therapeutic efficacy of TMZ in patients.

### DISCUSSION

This study represents the first systematic interrogation of all neurochemical classes on human GNS cell growth and proliferation. Of the 13 neurochemical classes tested, it was found that modulation of dopaminergic, serotonergic and cholinergic pathways predominantly affected GNS cells. It was further shown that DRD4 antagonists selectively inhibit the growth of GNS cells and reduce the colony forming potential of freshly dissociated GBM cells, both in vitro and in an in vivo patient-derived xenograft model. The selectivity of DRD4 antagonists such as L-741,742 and PNU 96415E for GNS cells is mediated by on-target inhibition of the DRD4 receptor, which is expressed in both GNS cells and primary glioblastoma patient samples, and concomitant suppression of the downstream effectors ERK1/2. At a cell biological level, DRD4 antagonism impairs a late step in the autophagy/lysosomal degradation pathway, resulting in massive accumulation of autophagic vacuoles, lysosomal cargo, and non-degraded ubiquitinated substrates. This effect is accompanied by a G₀/G₁ cell cycle arrest and non-apoptotic cell death.

Relevant portions of references referred to herein are incorporated by reference.

1. A method of treating a brain tumour in a mammal comprising administering to the mammal a dopamine receptor D₄ antagonist.

2. The method of claim 1, wherein the brain tumour is selected from the group consisting of glioblastoma multiforme, malignant astrocytoma, oligodendroglioma, oligoastrocytoma, mixed glioma, malignant glioma and medulloblastoma.

3. The method of claim 1, wherein the dopamine receptor D₄ antagonist is selected from the group consisting of A-381393, L-745,870, L-750,667, L-741,742, S 18126, fananserin, clozapine, buspirone, FAUC 213, sonepiprazole, PD 168568 dihydrochloride and PNU 96415E.

4. The method of claim 3, wherein the antagonist is L-741,742.

5. The method of claim 3, wherein the antagonist is PNU 96415E.

6. The method of claim 1, wherein the antagonist is administered at a dosage within the range of about 0.1-100 mg/m², or a dosage in the range of 1-100 mg/m², or a dosage in the range of 1-50 mg/m².

7. The method of claim 6, wherein the antagonist is formulated for infusion or injection.

8. The method of claim 7, wherein the antagonist is combined with a sterile aqueous solution in selected from distilled water, a carbohydrate-containing solution or a saline solution.

9. The method of claim 1, wherein the antagonist is administered in conjunction with an anti-neoplastic alkylating or alkylating-like agent.

10. The method of claim 10, wherein the anti-neoplastic alkylating or alkylating-like agent is selected from the group consisting of nitrogen mustards, nitrosoureas, alkyl sulfonates procarbazine, altretamine, triazines and platinum-based chemotherapeutic agents.

11. The method of claim 10, wherein the agent is selected from the group consisting of cyclophosphamide, chlorambucil, uramustine, ifosfamide, melphalan, bendamustinetriazenes, carmustine, lomustine, semustine, ethylnitrosourea (ENU), streptozocin, busulfan, dacarbazine, mitozolomide, temozolomide, cisplatin, carboplatin, nedaplatin, oxaliplatin, satraplatin and triplatin tetranitrate.

12. The method of claim 10, wherein the alkylating agent is a triazine.

13. The method of claim 12, wherein the alkylating agent is temozolomide.

14. The method of claim 9, wherein the antagonist is administered at a dosage in the range of about 0.1-50 mg/m² and the alkylating agent is administed at a dosage range of about 1-100 mg/m².

15. A synergistic composition comprising a dopamine receptor D₄ antagonist in combination with an anti-neoplastic alkylating or alkylating-like agent.

16. The composition of claim 15 wherein the agent is selected from the group consisting of nitrogen mustards, nitrosoureas, alkyl sulfonates procarbazine, altretamine, triazines and platinum-based chemotherapeutic agents.

17. The composition of claim 16, wherein the agent is selected from the group consisting of cyclophosphamide, chlorambucil, uramustine, ifosfamide, melphalan, bendamustinetriazenes, carmustine, lomustine, semustine, ethylnitrosourea (ENU), streptozocin, busulfan, dacarbazine, mitozolomide, temozolomide, cisplatin, carboplatin, nedaplatin, oxaliplatin, satraplatin and triplatin tetranitrate.

18. The composition of claim 16, wherein the alkylating agent is a triazine.

19. The composition of claim 18, wherein the alkylating agent is temozolomide.

20. The composition of claim 15, wherein the dopamine receptor D₄ antagonist is selected from the group consisting of A-381393, L-745,870, L-750,667, L-741,742, S 18126, fananserin, clozapine, buspirone, FAUC 213, sonepiprazole, PD 168568 dihydrochloride and PNU 96415E.

21. The composition of claim 15, wherein the antagonist is L-741,742.

22. The composition of claim 15, wherein the antagonist is PNU 96415E.

23. The composition of claim 15, comprising a dosage form having a dopamine receptor D₄ antagonist dosage of about 0.1-50 mg/m², and a dosage form having a temozolomide dosage of about 1-200 mg/m².

24. The composition of claim 15, which is formulated for infusion or injection.

25. The composition of claim 15, which is formulated for oral administration.
